(19) United States
(12) Patent Application Publication
(10) Pub. No.: US 2024/0044803 A1
(43) Pub. Date: Feb. 8, 2024

(54) ECL-BASED ELECTRODE, MANUFACTURING METHOD FOR ECL-BASED ELECTRODE, ECL SENSOR, MANUFACTURING METHOD FOR ECL SENSOR, METHOD FOR ECL DETECTION OF NUCLEIC ACID-SPECIFIC SITE MODIFICATION, AND KIT FOR NUCLEIC ACID MODIFICATION DETECTION USED FOR METHOD FOR ECL DETECTION OF NUCLEIC ACID-SPECIFIC SITE MODIFICATION

(71) Applicants: CANON MEDICAL SYSTEMS CORPORATION, Tochigi (JP); Nanjing University, Nanjing (CN)

(72) Inventors: Hao HUANG, Beijing (CN); Qiqi XU, Beijing (CN); Xiaotian WU, Beijing (CN); Huangxian JU, Nanjing (CN); Jie WU, Nanjing (CN)

(73) Assignees: CANON MEDICAL SYSTEMS CORPORATION, Tochigi (JP); Nanjing University, Nanjing (CN)

(21) Appl. No.: 18/361,335

(22) Filed: Jul. 28, 2023

(30) Foreign Application Priority Data
Jul. 28, 2022 (CN) 202210897954.1

(51) Int. Cl. G01N 21/66; G01N 21/76; G01N 33/543

(52) U.S. Cl. CPC G01N 21/66; G01N 21/76; G01N 33/5438

(57) ABSTRACT

A manufacturing method for an ECL (Electrochemiluminescence)-based electrode according to an embodiment includes a step of mixing an electrocatalytic solution and metal nanoparticles and thereafter dropping a mixed particle suspension onto the surface of an electrode and obtaining a base electrode co-modified by an electrocatalyst-metal nanoparticle.

### CROSS-REFERENCE TO RELATED APPLICATIONS

This application is based upon and claims the benefit of priority from Chinese Patent Application No. 202210897954.1, filed on Jul. 28, 2022, the entire contents of all of which are incorporated herein by reference.

Reference to Sequence Listing In accordance with 37 CFR § 1.833-1835 and 37 CFR § 1.77(b) (5), the specification makes reference to a Sequence Listing submitted electronically as a .xml file named “549194US_ST26.xml”. The .xml file was generated on Jul. 28, 2023 and is 10,017 bytes in size. The entire contents of the Sequence Listing are hereby incorporated by reference.

### FIELD

Embodiments disclosed in the present specification and the drawings relate to an ECL-based electrode, a manufacturing method for an ECL-based electrode, an ECL sensor, a manufacturing method for an ECL sensor, a method for ECL detection of a nucleic acid-specific site modification, and a kit for nucleic acid modification detection used in a method for ECL detection of a nucleic acid-specific site modification.

### BACKGROUND

A large volume of chemical modifications are present in nucleic acids, for example, a DNA and an RNA and these chemical modifications dynamically adjust gene expression as a regulatory mechanism based on the premise that a nucleic acid sequence is not changed.

DNA methylation is an apparent modification that occurs at a fifth carbon atom of cytosine and is stably inherited, and is widely present in animal and plant genomes. The DNA methylation is widely involved in various physiological processes including gene silencing, genomic imprinting, X-chromosome inactivation, and disease development during mammalian growth and development.

Since the development of a part of tumors is accompanied by hypermethylation events of specific genes and local hypermethylation events of genes occur earlier than malignant growth of cells, detection of methylation levels of specific genes can be an important basis for early tumor prediction and diagnosis. For example, early colorectal cancer (CRC) screening detection is performed based on apparent genetic biomarkers and the Food and Drug Administration (FDA) has already permitted to detect increased methylation of a specific gene promoter CpG to thereby perform early screening and adjunctive diagnosis of CRC (see Yvette N Lamb et al, Epi proColon® 2.0 CE: A Blood-Based Screening Test for Colorectal Cancer, Mol Diagn Ther. 2017 April; 21(2): 225-232). Different tumor types have different tumor suppressor genes that cause hypermethylation, for example, in ovarian cancer, tumor suppressor genes RASSF1A, BRCA1, APC, CDKN2A, and the like are hypermethylated and, in breast cancer, tumor suppressor genes PCDHB15, WBSCRF17, IGF1, GYPC, and the like exhibit a hypermethylated state (see Tingting Hong, Selective detections of epigenetic modification in DNA, Wuhan University, 2017, Ph.D dissertation). Detecting the methylation levels of these specific tumor suppressor genes not only enhances screening and diagnosis of specific tumors but also helps in the evaluation of the efficacy of definitive treatment for tumors and prognostic observation.

Currently, a standard method for DNA methylation detection is hydrogen sulfite treatment (bisulfite treatment). Specifically, treatment of DNA with hydrogen sulfite, for example, sodium hydrogen sulfite can convert cytosine (C) in DNA into uracil (U). In contrast, methylated 5-methylcytosine (5-mC) is retained unchanged. Consequently, 5-mC and C can be distinguished by the following PCR or sequencing and detection of methylated DNA can be realized.

However, hydrogen sulfite treatment requires pretreatment of nucleic acids under stringent chemical and temperature conditions, and the detection operation of PCR or sequencing is complex, requiring specialized technicians and specialized equipment, which makes the detection method inefficient, time consuming, and costly. Therefore, there is a need to develop a methylated DNA detection method that is simple, rapid, and sensitive.

Recently, a methylated DNA detection method by recognition of methylation sites by specific antibodies has been attracting attention. Since such a detection method does not require pretreatment of nucleic acids and specific antibodies are labelled with different probes, electrochemical (for example, Eloy Povedano et al., Amperometric Bioplatforms To Detect Regional DNA Methylation with Single-Base Sensitivity, Anal. Chem, 2020, 92, 5604-12), fluorescent, and other detection can be performed.

A recently developed ECL (Electrochemiluminescence) technology has already been widely applied in biological analyses, for example, detection of tumor protein-labeled substances (for example, Xiaoming Zhou et al., Synthesis, labeling and bioanalytical applications of a tris(2,2′-bipyridyl)Ruthenium(II)-based electrochemiluminescence probe, Nat Protoc 2014 May; 9(5)). The ECL technology is a method in which the electrochemical reaction is carried out at an electrode surface using an electrochemical principles to generate states of excitation, thereby producing emission of light. Since the ECL is electroluminescence, compared with fluorescence, it is unnecessary to add an excitation light source and there is no influence such as light fading and light interference. Therefore, the ECL has advantages such as simple equipment, low cost, low background signal, and high detection sensitivity.

However, there is a need to further improve detection performance such as sensitivity and specificity of the current ECL detection method.

One of the problems to be solved by embodiments disclosed in the present specification and the drawings is to provide a simple, sensitive, rapid, and general-purpose electrochemiluminescence detection method for nucleic acid-specific site modifications by employing an ECL nanoprobe and combining a discriminative reaction against methylation sites of specific antibodies. Based on this, the inventors have further intensively studied aspects such as enhancing an ECL detection signal, improving detection sensitivity and specificity, and the like, and as a result, have successfully manufactured an ECL-based electrode and a sensor having an interface enhancement effect by nanofunctionalized modification of the electrode surface and improved the sensitivity of ECL detection.

That is, the present embodiment can provide a method for ECL detection of nucleic acid-specific site modifications with high sensitivity, high specificity, and low detection limit through an ECL technology based on interface enhancement and a nanoprobe.

### BRIEF DESCRIPTION OF THE DRAWINGS

FIG. 1 is a schematic diagram illustrating manufacturing of an ECL-based electrode and an ECL sensor in an embodiment;

FIG. 2 is a schematic diagram illustrating manufacturing of an ECL nanoprobe used in the embodiment;

FIG. 3 is a schematic diagram illustrating ECL detection of nucleic acid-specific site modification using the ECL sensor in the embodiment;

FIG. 4 is a schematic diagram illustrating a mechanism of ECL interface enhancement in the embodiment;

FIG. 5A is a diagram illustrating a characterization result of a form of an ECL nanoprobe manufactured in an example 1;

FIG. 5B is a diagram illustrating a characterization result of the form of the ECL nanoprobe manufactured in the example 1;

FIG. 5C is a diagram illustrating a characterization result of the form of the ECL nanoprobe manufactured in the example 1;

FIG. 5D is a diagram illustrating a characterization result of the form of the ECL nanoprobe manufactured in the example 1;

FIG. 6 is a diagram illustrating a characterization result of ECL performance of the ECL nanoprobe manufactured in the example 1;

FIG. 7A is a diagram illustrating a measurement result of storage stability of the ECL nanoprobe manufactured in the example 1;

FIG. 7B is a diagram illustrating a measurement result of the storage stability of the ECL nanoprobe manufactured in the example 1;

FIG. 8A is a diagram illustrating an analysis result of the number of ruthenium molecules and the number of secondary antibodies bound on the ECL nanoprobe manufactured in the example 1;

FIG. 8B is a diagram illustrating an analysis result of the number of ruthenium molecules and the number of secondary antibodies bound on the ECL nanoprobe manufactured in the example 1;

FIG. 8C is a diagram illustrating an analysis result of the number of ruthenium molecules and the number of secondary antibodies bound on the ECL nanoprobe manufactured in the example 1;

FIG. 9A is a diagram illustrating a characterization result of a nanomaterial at an interface of an ECL sensor manufactured in an example 3;

FIG. 9B is a diagram illustrating a characterization result of the nanomaterial at the interface of the ECL sensor manufactured in the example 3;

FIG. 9C is a diagram illustrating a characterization result of the nanomaterial at the interface of the ECL sensor manufactured in the example 3;

FIG. 10A is a diagram illustrating a result of optimizing a construction condition of a sensor interface;

FIG. 10B is a diagram illustrating a result of optimizing the construction condition of the sensor interface;

FIG. 11A is a diagram illustrating electrochemical cyclic voltammetry (CV) and ECL measurement results in different modified electrodes;

FIG. 11B is a diagram illustrating electrochemical cyclic voltammetry (CV) and ECL measurement results in the different modified electrodes;

FIG. 11C is a diagram illustrating electrochemical cyclic voltammetry (CV) and ECL measurement results in the different modified electrodes;

FIG. 11D is a diagram illustrating electrochemical cyclic voltammetry (CV) and ECL measurement results in the different modified electrodes;

FIG. 12 is a diagram illustrating an evaluation result about selectivity of an ECL detection method in the embodiment;

FIG. 13A is a diagram illustrating an ECL response status under a different ECL detection condition of a sensor manufactured in the example 3;

FIG. 13B is a diagram illustrating an ECL response status under a different ECL detection condition of the sensor manufactured in the example 3;

FIG. 13C is a diagram illustrating an ECL response status under a different ECL detection condition of the sensor manufactured in the example 3;

FIG. 13D is a diagram illustrating an ECL response status under a different ECL detection condition of the sensor manufactured in the example 3;

FIG. 13E is a diagram illustrating an ECL response status under a different ECL detection condition of the sensor manufactured in the example 3;

FIG. 13F is a diagram illustrating an ECL response status under a different ECL detection condition of the sensor manufactured in the example 3;

FIG. 13G is a diagram illustrating an ECL response status under a different ECL detection condition of the sensor manufactured in the example 3;

FIG. 13H is a diagram illustrating an ECL response status under a different ECL detection condition of the sensor manufactured in the example 3;

FIG. 14A is a diagram illustrating a result of ECL detection of a target nucleic acid under optimal conditions;

FIG. 14B is a diagram illustrating a result of the ECL detection of the target nucleic acid under the optimal conditions;

FIG. 15 is a diagram illustrating a result of ECL detection of different sample solutions by an ECL detection method in the embodiment; and

FIG. 16 is a diagram illustrating a result of repeated detection of 10 pM target nucleic acid by sensors manufactured in different batches.

### DETAILED DESCRIPTION

A manufacturing method for an ECL-based electrode according to an embodiment includes a step of mixing an electrocatalytic solution and metal nanoparticles and thereafter dropping a mixed particle suspension onto a surface of an electrode and obtaining a base electrode co-modified by an electrocatalyst-metal nanoparticles.

A specific embodiment is explained in detail below with reference to the drawings. The following explanation is only to explain the inventive concept of the present embodiment and does not limit the present embodiment.

The present embodiment provides a method of electrochemiluminescence (ECL) detection for nucleic acid-specific site modification based on interface enhancement and a nanoprobe, a base electrode used in the ECL detection method, a sensor, and a manufacturing method therefor, a kit, and the like.

Detection of nucleic acid-specific site modification according to the present embodiment is realized by hybridization capture of nucleic acids and identification and labeling for a modification site, for example, a methylation site by a specific antibody and, therefore, does not require pretreatment and PCR amplification processes of nucleic acids in the detection method of the related art. A method is simple and detection efficiency is high. The present embodiment effectively improves an ECL signal because a base electrode is co-modified by an electrocatalyst and metal nanoparticles to construct an ECL-enhanced interface. The present embodiment greatly improves detection sensitivity of an ECL method with a double amplification policy combining interface enhancement and a nanoprobe. A detection limit for nucleic acid-specific site modification can reach up to an fM level. The present embodiment is versatile for detection of nucleic acid-specific site modification and can detect different target nucleic acid-specific site modifications by designing different capture nucleic acids and different specific antibodies.

ECL-Based Electrode and ECL Sensor

One embodiment relates to a manufacturing method for an ECL-based electrode including a step of mixing an electrocatalytic solution and metal nanoparticles and thereafter dropping a mixed particle suspension onto the surface of an electrode and obtaining a base electrode co-modified by an electrocatalyst-metal nanoparticles.

The electrocatalyst used in the present embodiment is not particularly limited and a two-dimensional nanomaterial can be used. The two-dimensional nanomaterial has been attracting attention because the two-dimensional nanomaterial has satisfactory mechanical, thermal, and electronic performance and high catalytic activity. The two-dimensional nanomaterial specifically may be a transition metal compound and is, for example, a transition metal disulfide. Specific examples of the transition metal disulfide include a molybdenum disulfide (MoS₂), a tungsten disulfide (WS₂), molybdenum diselenide (MoSe₂), and a tungsten diselenide (WSe₂). Among them, the molybdenum diselenide (MoSe₂) is preferably used.

The metal nanoparticles used in the present embodiment are not particularly limited and are, for example, at least one of gold (Au) nanoparticles, silver (Ag) nanoparticles, platinum (Pt) nanoparticles, copper (Cu) nanoparticles, cobalt (Co) nanoparticles, iron (Fe) nanoparticles, nickel (Ni) nanoparticles, and multiple alloy nanoparticles thereof. Among them, the gold (Au) nanoparticles are preferably used.

The electrocatalyst and the metal nanoparticles used in the present embodiment can be manufactured when the base electrode is manufactured. Commercially available products can be used as they are.

The electrode used in the present embodiment is not particularly limited. One of common electrodes in ECL detection, for example, a glass carbon electrode (GCE), an indium tin oxide (ITO) electrode, and a screen printed electrode (SPE) can be used. Among them, the glass carbon electrode (GCE) is preferably used. More preferably, a standard three-electrode system in which a platinum wire electrode, an Ag/AgCl reference electrode, and a glass carbon working electrode (GCE) are arranged is used.

In terms of obtaining higher ECL signal intensity, preferably, construction conditions for an interface of a base electrode, for example, an electrocatalyst exfoliation time, a mixing ratio of an electrocatalyst and metal nanoparticles, and the like are optimized.

The electrocatalyst exfoliation time refers to a time for dispersing, with ultrasound, a synthesized transition metal disulfide into a two-dimensional nanosheet-like structure. The electrocatalyst exfoliation time is not particularly limited as long as a uniform two-dimensional nanosheet of a transition metal disulfide can be obtained. For example, the electrocatalyst exfoliation time may be five hours or longer, preferably five to 25 hours, more preferably 10 to 20 hours, and particularly preferably 10 hours.

The mixing ratio of the electrocatalyst and the metal nanoparticles is not particularly limited as long as the ECL-based electrode in the present embodiment can be obtained. For example, the mixing ratio may be 1:1 to 1:9 in a volume ratio of 3.5 mg/mL of MoSe₂ and 0.5 mg/mL of AuNPs and is preferably 1:5 to 1:7 and is more preferably 1:6.

Consequently, the present embodiment further relates to an ECL-based electrode manufactured by the manufacturing method explained above. The ECL-based electrode is co-modified by an electrocatalyst and metal nanoparticles. In the present embodiment, the electrocatalyst can be directly bound to an ECL electrode by being dispersed into a two-dimensional nanosheet-like structure. In other words, in the present embodiment, the electrocatalyst can be directly bound to the ECL electrode not via another layered structure, for example, graphene. Therefore, the present embodiment can more conveniently and easily obtain a base electrode for ECL detection.

One embodiment further relates to a manufacturing method for an ECL sensor including a step of manufacturing an ECL-based electrode with the manufacturing method explained above and a step of binding a modified capture nucleic acid to the surface of the base electrode. In other words, the manufacturing method for the ECL sensor includes a step of manufacturing an ECL-based electrode, with a manufacturing method for an ECL-based electrode including a step of mixing an electrocatalytic solution and metal nanoparticles and thereafter dropping a mixed particle suspension onto the surface of an electrode and obtaining a base electrode co-modified by electrocatalyst-metal nanoparticles, and a step of binding a terminal-modified capture nucleic acid to the surface of the base electrode.

The capture nucleic acid is designed for a target nucleic acid and has a nucleic acid sequence complementary to the target nucleic acid. The capture nucleic acid and the target nucleic acid can be stably and specifically hybridized. The capture nucleic acid can be synthesized by a general method in this field or a commercially available commercial product can be used as is. The capture nucleic acid is modified in order to enable the capture nucleic acid to be fixed to the base electrode via a modification group. As a modification method, general various modification methods in this field can be employed. The modification method is not particularly limited. For example, mercapto modification, amino modification, biotinylation modification, and the like can be employed. Among them, the mercapto modification is preferable because the mercapto modification is the most commonly used. In other words, the terminal-modified capture nucleic acid is a mercapto-modified capture nucleic acid, an amino-modified capture nucleic acid, or a biotinylated capture nucleic acid.

The manufacturing method for the ECL sensor in the present embodiment may further include a step of blocking, with a blocking agent, the surface of the electrode to which the capture nucleic acid is bound. Specifically, after binding the capture nucleic acid to the electrode, a non-specific site on the surface of the electrode is blocked by the blocking agent. The blocking agent is not particularly limited and a general blocking agent in this field can be employed. Preferably, at least one of a small molecule blocking agent and a protein blocking agent is used in terms of a method of constructing the surface of a DNA sensor and the surface of a protein sensor generally used in this field.

As the small molecule blocking agent, for example, 6-mercapto-1-hexanol (MCH), mercaptoethylene glycol (HS—(CH₂)₁₁-EG₂-OH, OEG), mercaptopropionic acid (MPA), polyethylene glycol (PEG), and polyethyleneimine (PEI) can be used. Among them, the 6-mercapto-1-hexanol (MCH) is preferably used. As the protein blocking agent, for example, bovine serum albumin (BSA), animal serum, nonfat milk powder, heparin, salmon sperm DNA, and the like can be used. Among them, the bovine serum albumin (BSA) is preferably used.

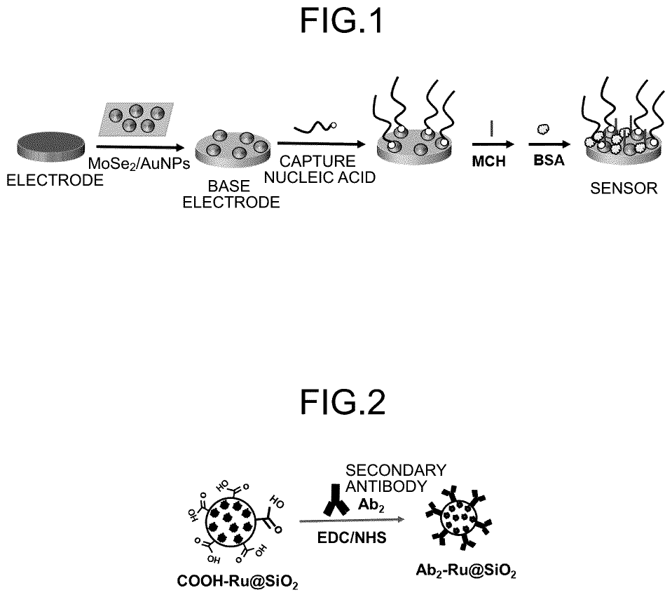

FIG. 1 is a schematic diagram illustrating manufacturing of an ECL-based electrode and an ECL sensor in the present embodiment and is a manufacturing example in which molybdenum diselenide (MoSe₂) is used as an electrocatalyst and gold (Au) nanoparticles (AuNPs) are used as metal nanoparticles.

As illustrated in FIG. 1, first, molybdenum diselenide (MoSe₂) and gold (Au) nanoparticles (AuNPs) are prepared. The molybdenum diselenide (MoSe₂) and gold (Au) nanoparticles (AuNPs) are mixed and thereafter dropped onto an electrode to obtain a base electrode co-modified by MoSe₂/AuNPs.

Further, a modified capture nucleic acid is added to the surface of the base electrode co-modified by MoSe₂/AuNPs and the capture nucleic acid is bound to the surface of the base electrode to obtain an ECL-based electrode to which the capture nucleic acid is bound, that is, the ECL sensor in the present embodiment. In other words, the ECL sensor includes the ECL-based electrode having an interface co-modified by an electrocatalytic solution and metal nanoparticles and the terminal-modified capture nucleic acid bound to the surface of the ECL-based electrode.

The surface of the ECL sensor obtained as explained above may be further blocked by a blocking agent, for example, MCH, which is a small molecule blocking agent, and/or BSA, which is a protein blocking agent. Preferably, the blocking is performed sequentially using the MCH and the BSA.

The ECL-based electrode and the ECL sensor in the present embodiment may be manufactured prior to detection or at the time of detection.

In the present embodiment, interface enhancement design for the electrocatalyst and the metal nanoparticles explained above can effectively promote electron transfer on an electrode surface in an electrochemiluminescence system and enhance an ECL detection signal. As explained in examples below in the present embodiment, a molybdenum diselenide nanomaterial (MoSe₂)/gold nanoparticles (Au NPs) can effectively promote electron transfer and a tris(bipyridine)ruthenium(II) complex ion-tripropylamine (Ru(bpy)₃²⁺-TPrA) system has an ECL-enhancing effect. Consequently, it is possible to improve detection sensitivity of a sensor.

Consequently, one embodiment relates to an ECL sensor manufactured by the manufacturing method explained above.

ECL Detection Method for Nucleic Acid-Specific Site Modification

One embodiment relates to an ECL detection method for nucleic acid-specific site modification including a step 1 of using an ECL sensor manufactured by the manufacturing method explained above, dropping a test sample onto the surface of the ECL sensor, and capturing a target nucleic acid on the surface of the sensor with a capture nucleic acid, a step 2 of dropping an anti-nucleic acid modification antibody and binding the anti-nucleic acid modification antibody to a nucleic acid-specific site on the target nucleic acid, a step 3 of adding an ECL nanoprobe to label the target nucleic acid with a detection signal, and a step 4 of placing the sensor obtained in the step 3 in a detection solution containing a co-reactive agent and performing ECL signal measurement. That is, the ECL detection method for nucleic acid-specific site modification includes a first step of dropping a test sample onto the surface of an ECL sensor including an ECL-based electrode having an interface co-modified by an electrocatalytic solution and metal nanoparticles and the terminal-modified capture nucleic acid bound to the surface of the ECL-based electrode and capturing a target nucleic acid on the surface of the ECL sensor with the capture nucleic acid, a second step of dropping an anti-nucleic acid modification antibody and binding the anti-nucleic acid modification antibody to a nucleic acid-specific site on the target nucleic acid, a third step of adding an ECL nanoprobe and labelling the target nucleic acid with a detection signal, and a fourth step of immersing the ECL sensor obtained in the third step in a detection solution containing a co-reactive agent and performing ECL signal measurement.

The present embodiment detects nucleic acid-specific site modification with an electrochemiluminescence method. The nucleic acid is, for example, a DNA or an RNA. The nucleic acid-specific site modification is not particularly limited and may be, for example, methylation modification, methylolation modification, or formylation modification of a nucleic acid and specifically is, for example, DNA methylation modification (for example, methylation of 5-methylcytosine, hereinafter also referred to as 5mC), methylolation modification (for example, methylolation of 5-methylcytosine, hereinafter also referred to as 5hmC), formylation modification (for example, formylation of 5-cytosine, hereinafter also referred to as 5fmC), RNA methylation modification (for example, methylation of the sixth N of adenine, hereinafter also referred to as m6A), and the like.

An electrochemiluminescence system is mainly divided into two types according to luminescent reagents: (1) a metal complex electrochemiluminescence system and (2) an organic compound electrochemiluminescence system including fused ring aromatic hydrocarbons and hydrazides. A regularly used electrochemiluminescence metal complex includes metal ion complexes such as Ru, Os, Re, Ir, Cr, Pd, Al, Cd, Pt, Mo, Tb, and Eu. Among them, the metal complexes of Ru, Ir, Os, and Re have been attracting attention because the metal complexes have satisfactory electrochemiluminescence properties.

In the Ru metal complex, a tris(bipyridine)ruthenium(II) complex ion (Ru(bpy)₃²⁺) is widely applied because of characteristics such as high water solubility, stable chemical performance, reversible redox, high luminescence efficiency, wide pH range for application, electrochemical regenerability, and long excited state lifetime. Here, Ru(bpy)₃²⁺ reacts with tripropylamine (TPrA), which is a co-reactive agent, to enable ECL detection under low potential conditions. A reaction formula between Ru(bpy)₃²⁺ and tripropylamine TPrA is as follows.

Tripropylamine→Tripropylamine·+e− (1)

Ru(bpy)₃²⁺→Ru(bpy)₃³⁺+e− (2)

Ru(bpy)₂³⁺Tripropylamine·→[Ru(bpy)₃²⁺]*+product (3)

[Ru(bpy)₃²⁺]*→Ru(bpy)₃²⁺+hv (4)

The electrochemiluminescence detection method in the present embodiment is preferably performed by a ruthenium (II) complex ion system, but can also be performed by using another metal complex ion system, for example, an iridium (III) complex ion system.

The anti-nucleic acid modification antibody is a specific antibody against specific site modification of a target nucleic acid (hereinafter also referred to as primary antibody). The antibody may be prepared by a general preparation method for antibodies in this field according to specific site modification or a commercially available product may be used as it is. The primary antibody is, for example, an antibody against DNA methylated cytosine (5mC) or methylol cytosine (5hmC).

As an electrochemiluminescence (ECL) nanoprobe, various ECL nanoprobes used in this field can be used. However, preferably, an electrochemiluminescence nanoprobe described in Chinese Patent Application 202210592343.6 filed by the applicant before the present application (hereinafter also referred to as ECL nanoprobe), that is, metal-doped inorganic oxide nanoparticles modified by a secondary antibody is used.

The method of preparing the ECL nanoprobe includes a step of adding metal complex ions to inorganic oxide nanoparticles to obtain metal-added inorganic oxide nanoparticles and a step of binding a secondary antibody to the metal-added inorganic oxide nanoparticles to obtain secondary antibody-modified metal-added inorganic oxide nanoparticles, the secondary antibody being affinity-bound to the anti-nucleic acid modification antibody.

For the preparation of the ECL nanoprobe, a nanomaterial is used as a carrier. Since the nanomaterial has a huge surface area or porous structure, the nanomaterial can be used for ultrasensitive detection by carrying a large volume of a luminescent material. In terms of high stability, low cost, and ease of modification, inorganic oxide nanoparticles are preferably used as the nanomaterial used in the present embodiment. Examples of the inorganic oxide nanoparticles specifically include silicon dioxide nanoparticles, titanium dioxide nanoparticles, zinc oxide nanoparticles, and iron oxide nanoparticles, or nanoparticles coated with silicon dioxide, titanium dioxide, zinc oxide, iron oxide, and the like on the surfaces. Among them, the silicon dioxide nanoparticles are more preferable. That is, the inorganic oxide nanoparticles are nanoparticles coated with silicon dioxide, titanium dioxide, zinc oxide, or iron oxide, silicon dioxide nanoparticles, titanium dioxide nanoparticles, zinc oxide nanoparticles, or iron oxide nanoparticles.

The method of preparing the ECL nanoprobe includes a step of adding metal complex ions to inorganic oxide nanoparticles. As an addition method, a normally-used addition method in this field, for example, an electrochemical method, a sol-gel method, an ion-exchange method, and a hydrolytic precipitation method can be used. However, the addition method is not limited to these methods. Any method that can add metal complex ions to inorganic oxide nanoparticles can be used.

The method of preparing the ECL nanoprobe further includes a step of binding a secondary antibody to the metal-added inorganic oxide nanoparticles to obtain secondary antibody-modified metal-added inorganic oxide nanoparticles. Here, a binding method for the secondary antibody and the metal-added inorganic oxide nanoparticles is preferably performed by a covalent binding method. However, other methods such as electrostatic adsorption can also be used. Examples of a binding method for forming covalent binding specifically include carboxy-amino binding and aldehyde group-amino binding. Reaction to form covalent binding is not particularly limited but is preferably a covalent binding reaction that can react at room temperature, is rapid, and has high binding efficiency.

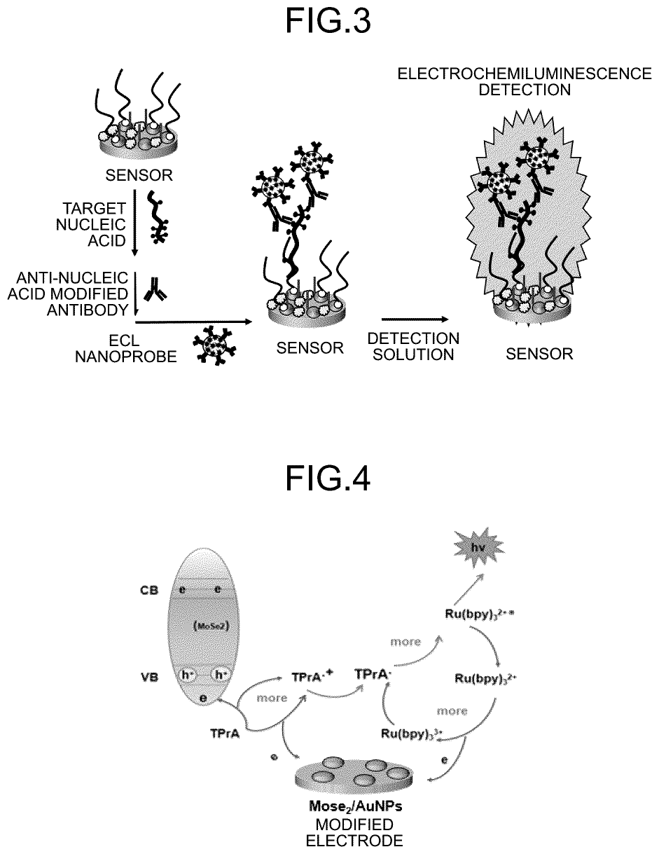

FIG. 2 is a schematic diagram illustrating manufacturing of the ECL nanoprobe used in the present embodiment. In FIG. 2, a tris(bipyridine)ruthenium(II) complex ion (Ru(bpy)₃²⁺) is used as an example and an EDC/NHS system (1-(3-dimethylaminopropyl)-3-ethylcarbodiimide hydrochloride)/N-hydroxysuccinimide system) is used to bind the secondary antibody (hereafter also referred to as Ab₂) to the metal-added inorganic oxide nanoparticles. Specifically, Ru(bpy)₃²⁺ is added to silicon dioxide nanoparticles to obtain ruthenium-doped silicon dioxide nanoparticles (hereafter also referred to as Ru@SiO₂). Subsequently, Ru@SiO₂ is carboxylated to form carboxylated ruthenium-added silicon dioxide nanoparticles (hereinafter also referred to as COOH—Ru@SiO₂). Subsequently, the secondary antibody is bound to COOH—Ru@SiO₂ using the EDC/NHS system to form secondary antibody-modified ruthenium-added silicon dioxide nanoparticles (hereafter also referred to as Ab₂-Ru@SiO₂).

Since the ECL nanoprobe can bind a large volume of metal complex ions per one nanobead, an electrochemical signal can be markedly amplified. Consequently, it is possible to greatly improve sensitivity of ECL detection.

In the ECL nanoprobe, a plurality of secondary antibody molecules can be bound per one nanobead. Consequently, it is possible to improve binding efficiency between a nanoprobe and a primary antibody.

The ECL nanoprobe prepared by the method explained above is excellent in storage stability and can be stored for 10 days or more under, for example, a storage condition at room temperature. Therefore, the ECL nanoprobe used in the present embodiment can be used as it is after preparation but can also be used when necessary after being stored for a certain time.

The secondary antibody identifies the primary antibody against nucleic acid-specific site modification. The secondary antibody is preferably not designed for a binding specific portion of the primary antibody and nucleic acid-specific site modification but is designed for a general-purpose portion of the primary antibody. That is, the secondary antibody used in the present embodiment is preferably protein that identifies the general-purpose portion of the primary antibody. The secondary antibody is not particularly limited and can be prepared by a general antibody preparation method in this field, or a commercially available product can be directly used. The general-purpose portion is also referred to as stationary region.

Consequently, the present embodiment can greatly improve detection sensitivity of the ECL method with a double amplification policy in which interface enhancement and a nanoprobe are combined, and reduce a detection limit of the nucleic acid-specific site modification to the fM level.

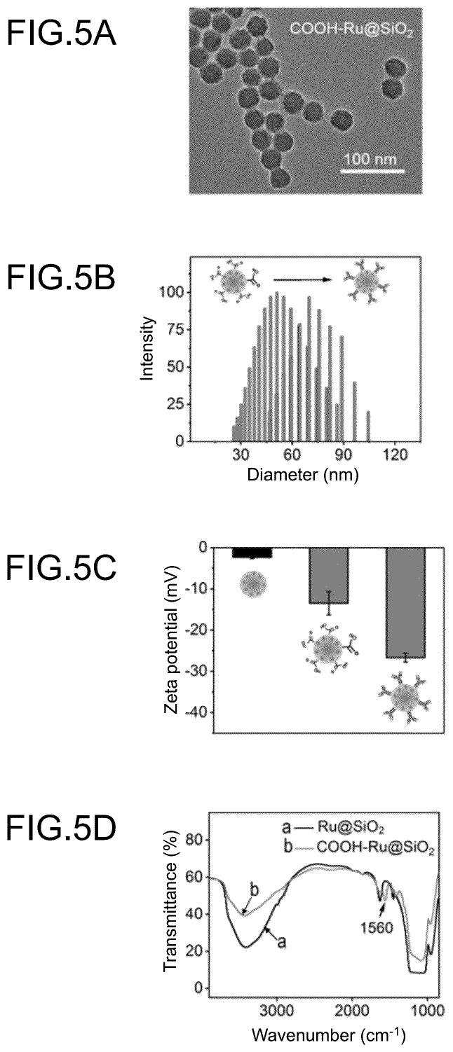

FIG. 3 is a schematic diagram illustrating ECL detection of the nucleic acid-specific site modification using the ECL sensor in the present embodiment.

As illustrated in FIG. 3, a test sample likely to contain a target nucleic acid is dropped onto the ECL sensor in the present embodiment and a capture nucleic acid captures the target nucleic acid on the surface of the base electrode of the sensor with specific hybridization. Subsequently, an anti-nucleic acid modification antibody is added and the antibody binds to a nucleic acid-specific site on the target nucleic acid. Subsequently, an ECL nanoprobe is added to a sensor interface and the target nucleic acid is labeled with a detection signal by identification of the secondary antibody in the nanoprobe to the anti-nucleic acid modification antibody. A sensor obtained as explained above is placed in a detection solution containing a co-reactive agent and ECL signal measurement is performed.

In terms of obtaining a high ECL signal, ECL detection conditions are preferably optimized. Examples of the ECL detection conditions include pH of the ECL detection solution, concentration of the capture nucleic acid, an incubation time of the capture nucleic acid, an incubation time of the target nucleic acid, concentration of the primary antibody, an incubation time of the primary antibody, concentration of the ECL nanoprobe, and an incubation time of the ECL nanoprobe.

The pH of the ECL detection solution may be 6.0 or higher, preferably 6.5 to 8.5, and more preferably 7.5 to 8.0, and may be specifically 6.0, 6.5, 7.0, 7.5, 8.0, 8.5, and the like.

The concentration of the capture nucleic acid may be 1 nM to 400 nM, preferably 20 nM to 100 nM, and more preferably 30 nM, and may be specifically, for example, 1 nM, 10 nM, 30 nM, 40 nM, 50 nM, 60 nM, 70 nM, 80 nM, 90 nM, 100 nM, 200 nM, 300 nM, 400 nM, and the like.

The capture nucleic acid incubation time may be five minutes or longer, preferably 10 minutes or longer, more preferably 20 minutes or longer, and still more preferably 50 minutes, and may be specifically, for example, 10 minutes, 20 minutes, 30 minutes, 40 minutes, 50 minutes, 60 minutes, and the like.

The incubation time of the target nucleic acid may be five minutes or longer, preferably 10 minutes or longer, more preferably 20 minutes or longer, and still more preferably 30 minutes, and may be specifically, for example, 10 minutes, 20 minutes, 30 minutes, 40 minutes, 50 minutes, 60 minutes, and the like.

The concentration of the primary antibody may be 1 μg/mL or higher, preferably 2 μg/mL or higher, and more preferably 3 μg/mL, and may be specifically, for example, 1 μg/mL, 2 μg/mL, 3 μg/mL, 4 μg/mL, 5 μg/mL, 6 μg/mL, 7 μg/mL, 8 μg/mL, and the like.

The incubation time of the primary antibody may be five minutes or longer, preferably 10 minutes or longer, more preferably 20 minutes or longer, and still more preferably 40 minutes, and may be specifically, for example, five minutes, 10 minutes, 20 minutes, 30 minutes, 40 minutes, 50 minutes, 60 minutes, and the like.

The concentration of the ECL nanoprobe may be 1 μg/mL or higher, preferably 2 μg/mL or higher, more preferably 6 μg/mL to 12 μg/mL, still more preferably 8 μg/mL to 10 μg/mL, and most preferably 8 μg/mL, and may be specifically, for example, 2 μg/mL, 3 μg/mL, 4 μg/mL, 5 μg/mL, 6 μg/mL, 7 μg/mL, 8 μg/mL, 9 μg/mL, 10 μg/mL, 11 μg/mL, 12 μg/mL, and the like.

The incubation time of the ECL nanoprobe may be five minutes or longer, preferably 10 minutes or longer, more preferably 20 minutes or longer, and still more preferably 30 minutes or longer, and may be specifically, for example, 10 minutes, 20 minutes, 30 minutes, 40 minutes, 50 minutes, 60 minutes, and the like.

In the present embodiment, by using the ECL nanoprobe and the ECL sensor, it is possible to enhance a detection signal of an ECL and greatly improve detection sensitivity of the ECL. As explained in the Examples of the present embodiment, the inventors of the present embodiment have diligently studied a mechanism of ECL interface enhancement in the present embodiment and infer the mechanism as follows.

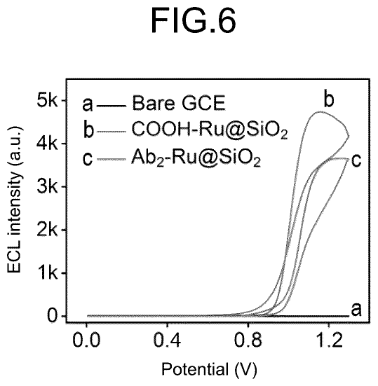

FIG. 4 is a schematic diagram illustrating the mechanism of the ECL interface enhancement in the present embodiment. As illustrated in FIG. 4, AuNPs on a sensor interface can promote the oxidation generation of Ru(bpy)₃³⁺ by facilitating electron transfer between Ru(bpy)₃²⁺ and a base electrode. On the other hand, MoSe₂ on the sensor interface lowers an oxidation level of TPrA, promotes TPrA to lose electrons, and generates radicals (TPrA·). Therefore, Ru(bpy)₃³⁺ and TPrA· are efficiently generated in the sensor interface, Ru(bpy)₃³⁺ and TPrA· can further react to generate Ru(bpy)₃²⁺ in an excited state and generate an ECL signal. Efficient generation of Ru(bpy)₃³⁺ and TPrA· in an AuNPs/MoSe₂/GCE sensing electrode is helped, Ru(bpy)₃²⁺ can also be efficiently generated, and an enhanced ECL signal is finally obtained.

One embodiment relates to a kit for nucleic acid modification detection used for the ECL detection method in the present embodiment, the kit including the ECL-based electrode or the ECL sensor in the present embodiment, a capture nucleic acid modified according to necessity, an anti-nucleic acid modification antibody, an ECL nanoprobe, and a detection solution.

In addition to the ECL-based electrode, the ECL sensor, the anti-nucleic acid modification antibody, and the ECL nanoprobe explained above, the kit can include various reagents necessary according to purpose of use, for example, a blocking agent and an instruction manual. That is, the kit for nucleic acid modification detection used for the ECL detection method for nucleic acid-specific site modification includes the ECL-based electrode having an interface co-modified by an electrocatalytic solution and metal nanoparticles, the terminal-modified capture nucleic acid bound to the surface of the ECL-based electrode, the anti-nucleic acid modification antibody, the ECL nanoprobe, and the detection solution.

Qualitative and quantitative analyses can be performed for nucleic acid-specific site modification for a sample detected based on presence or absence and intensity of an ECL signal, for example, when an ECL signal is not detected in a reaction system, it is determined that nucleic acid-specific site modification is not included in the sample to be detected, the complex is not formed, and an electrochemiluminescence signal cannot be generated. As verified in the examples of the present embodiment, since a logarithm of the intensity of electrochemiluminescence and the concentration of methylated DNA exhibits a linear relation, a quantitative analysis of the methylated DNA can be performed based on the intensity of the electrochemiluminescence.

### EXAMPLES

The present embodiment is specifically explained below with reference to examples. However, these examples are only illustrations and the present embodiment is not limited to these examples.

All reagents used in the examples are at an analytical purity level and sales destinations and abbreviations of the reagents are as explained below.

Tris(2,2′-bipyridine)dichlororuthenium (II) hexahydrate (Ru(bpy)₃²⁺), Triton X-100, Tween-20, tetraethyl orthosilicate (TEOS) and 2-(N-morpholine) ethanol sulfonic acid (MES) are purchased from Sigma (Shanghai, China).

Carboxyethylsilanetriol Na salt (CTES), N-(3-dimethylaminopropyl)-N′-ethylcarbodiimide hydrochloride (EDC), N-hydroxysuccinimide (NHS), and tri-n-propylamine (TPrA) are purchased from J&K Scientific (Shanghai, China).

Cyclohexane is purchased from Aladdin (Shanghai, China).

n-Hexanol is purchased from TCI chemicals (Shanghai, China).

Ammonia water is purchased from Macklin (Shanghai, China).

Acetone is purchased from Guo Yaku (Shanghai, China).

Bovine serum albumin (BSA) is purchased from KeyGEN BioTECH (Jiangsu, China).

Anti 5-methylcytosine antibody (product number: ab10805, Ab-5mC) and rabbit anti-mouse IgG antibody (product number: ab 6709, Ab₂) are purchased from Abcam (Shanghai, China).

A Bradford method protein quantitative detection kit is purchased from Sangon Biotech (Shanghai, China).

Ultrapure water is obtained from a water purification system (resistivity ≥18 MΩ·cm) of Millipore Corporation.

### Example 1: Preparation of ECL Nanoprobe (Ab₂-Ru@SiO₂)

Ru@SiO₂, COOH—Ru@SiO₂ and Ab₂-Ru@SiO₂ are respectively prepared according to the following steps.

First, a 50 mL round bottom flask is placed on a magnetic stirrer, high-speed magnetic stirring is applied, 7.50 mL of cyclohexane, 1.80 mL of n-hexanol, and 1.77 mL of Triton X-100 are added in order and mixed and stirred for 15 minutes. Subsequently, 340 μL of a 40 mM [Ru(byp)₃]²⁺ aqueous solution was added to a mixture of the above and mixed and stirred for 30 minutes. Thereafter, 100 μL of TEOS was added and stirred for 30 minutes. Subsequently, 60 μL of ammonia water (28 to 30% wt) was added to the mixture and stirred for 24 h under a light blocking condition to obtain Ru@SiO₂.

Subsequently, 50 μL of CTES (for carboxyl group modification) was added and caused to react for 7 h under the light blocking condition to obtain COOH—Ru@SiO₂. Thereafter, precipitate was separated by acetone (10,000 rpm, 10 minutes), a supernatant was centrifuged to be discarded, the precipitate was washed twice with ethanol and ultrapure water, respectively, dispersed by ultrasound, and the supernatant was centrifuged to be removed. Finally, the obtained precipitate is dispersed in ultrapure water, a part of the precipitate is taken, dried, and weighted and the remainder is stored at 4° C. under the light blocking condition.

A 10 μL of 50 mg/mL COOH—Ru@SiO₂ solution is put in a 1.5 mL centrifuge tube, added with 500 μL of EDC/NHS solutions (both of the EDC and NHS solutions are 50 mg/mL and prepared into 25 mM of a pH 5.5 MES buffer solution), vibrated for 35 minutes at the room temperature, thereafter, centrifugally separated to remove a supernatant, and, subsequently, centrifuged and cleaned (12,000 rpm, five minutes) by 10 mM of PH 7.4 PBS (phosphate buffer: prepared in NaH₂PO₄ and Na₂HPO₄). Thereafter, carboxylated COOH—Ru@SiO₂ is dispersed in 1 mL of 10 mM PBS, added with 20 μL of 2 mg/mL Ab₂, placed on a rocking bed (60 rpm), and caused to react for 4 h at the room temperature. Thereafter, carboxylated COOH—Ru@SiO₂ was centrifuged and cleaned (12,000 rpm, five minutes) at 4° C. using 25 mM of MES and 10 mM of PBS in order and a supernatant was discarded to obtain Ab₂-Ru@SiO₂. Finally, the Ab₂-Ru@SiO₂ is dispersed in 10 mM of PH7.4 PBS containing 2% of BSA and 0.05% of Tween-20 and stored at 4° C. under the light blocking condition.

### Example 2: Characterization of the ECL Nanoprobe Manufactured in the Example 1

(1) Characterization of a Form of the Nanoprobe

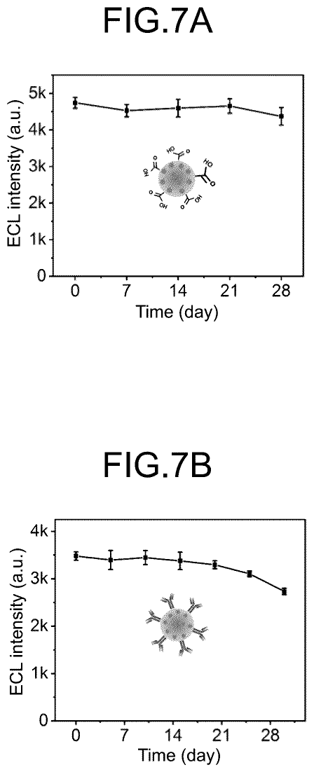

Forms of Ru@SiO₂, COOH—Ru@SiO₂, and Ab₂-Ru@SiO₂ nanoprobes obtained in the example 1 were respectively measured using a transmission electron microscope, a dynamic light scattering method, a Zeta potential measurement method, and an infrared (FT-IR) spectroscopy. Results of the measurement are illustrated in FIGS. 5A to 5D. FIGS. 5A to 5D are diagrams illustrating characterization results of a form of the ECL nanoprobe produced in the example 1. FIG. 5A illustrates a TEM photograph of COOH—Ru@SiO₂ obtained in the example 1. FIG. 5B illustrates DLS particle size distributions of COOH—Ru@SiO₂ and Ab₂-Ru@SiO₂. FIG. 5C illustrates measurement results of Zeta potential of Ru@SiO₂, COOH—Ru@SiO₂, and Ab₂-Ru@SiO₂ produced in the example 1. FIG. 5D illustrates measurement results of FT-IR spectra of COOH—Ru@SiO₂ (a) and Ab₂-Ru@SiO₂ (b) produced in the example 1.

A: Check of COOH—Ru@SiO₂ Formation by TEM

COOH—Ru@SiO₂ nanoparticles obtained in the example 1 were photographed by a JEM-2100 transmission electron microscope (JEOL, Japan). An obtained transmission electron micrograph (TEM) is illustrated in FIG. 5A.

As illustrated in FIG. 5A, the obtained COOH—Ru@SiO₂ nanoparticles are spherical particles uniformly dispersed, having a uniform size, and having a diameter of approximately 30 nm.

B: Check of Ab₂-Ru@SiO₂ Formation by a Dynamic Light Scattering (DLS) Method

Hydrated particle sizes of COOH—Ru@SiO₂ and Ab₂-Ru@SiO₂ were respectively measured by a dynamic light scattering (DLS) method using 90 Plus/BI-MAS equipment (Brookhaven, USA). A result of a particle size distribution is illustrated in FIG. 5B.

As illustrated in FIG. 5B, a hydrated particle size of COOH—Ru@SiO₂ (a) is distributed at approximately 50 nm, having a narrow particle size distribution, having a uniform size, and having monodispersity. A hydrated particle size of Ab₂-Ru@SiO₂ (b) is approximately 70 nm, which is slightly larger than that of COOH—Ru@SiO₂ and indicates that coupling between Ab₂ and Ru@SiO₂ are successful, dispersibility is high, and an agglomeration phenomenon does not occur.

C: Check of Ab₂-Ru@SiO₂ Formation by Zeta Potential Measurement

Zeta potential of the Ru@SiO₂, COOH—Ru@SiO₂, and Ab₂-Ru@SiO₂ nanoprobes produced in the example 1 were measured using 90 Plus/BI-MAS equipment (Brookhaven, USA). Results of the measurement are illustrated in FIG. 5C.

A change in the Zeta potential indicates that Ru@SiO₂ generated carboxylation and antibody binding. As illustrated in FIG. 5C, a Zeta potential value of Ru@SiO₂ is −2.1 mV and, since a carboxyl functional group has a negative charge, potential of COOH—Ru@SiO₂ is −11.6 mV. Since an antibody has a negative charge, a Zeta potential value of Ab₂-Ru@SiO₂ increases to −25.5 mV and indicates that Ab₂-Ru@SiO₂ was successfully produced.

D: Check of Ru@SiO₂ Carboxylation Modification by Infrared Spectra

Infrared (FT-IR) spectra of Ru@SiO₂ and COOH—Ru@SiO₂ produced in the example 1 were measured using a ThermoFisher Nicolet 6700 Fourier infrared spectrometer (Thermo, USA). Results of the measurement are illustrated in FIG. 5D.

Changes in the infrared spectra of Ru@SiO₂ and COOH—Ru@SiO₂ indicate that Ru@SiO₂ is carboxylation-modified. As illustrated in FIG. 5D, both of Ru@SiO₂ and COOH—Ru@SiO₂ have strong peaks around 1090 cm⁻¹. This is a characteristic peak of Si—O—Si tensile vibration. COOH—Ru@SiO₂ has an absorption peak by a new carboxyl group at 1560 cm⁻¹.

(2) ECL Performance Characterization of the ECL Nanoprobe

ECL detection was performed for COOH—Ru@SiO₂ and Ab₂-Ru@SiO₂ produced in the example 1 as explained below using an electrochemical cyclic voltammetry (CV) method.

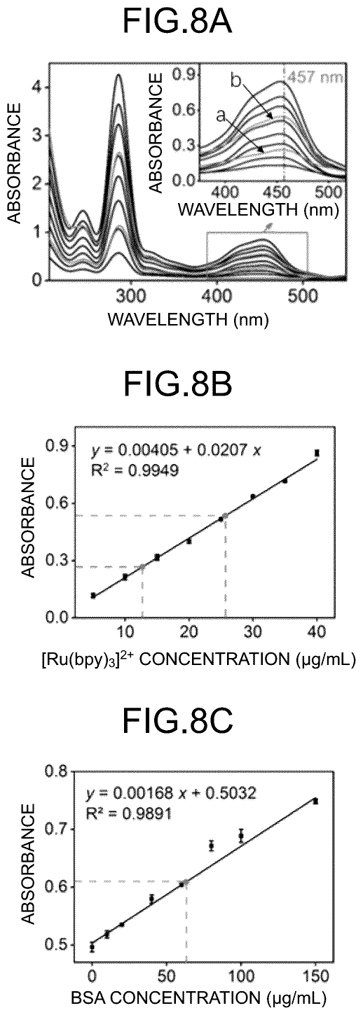

10 μL of COOH—Ru@SiO₂ and Ab₂-Ru@SiO₂ were respectively placed on glass carbon electrodes (GCE), dried, and thereafter, in an ECL detection solution (containing 0.1 M of PH7.4 PBS and 10 mM of TPrA), electrochemical cyclic voltammetry (CV) was performed at 0 V to +1.3 V using a three electrode system, and ECL intensity was acquired from a photomultiplier tube (PMT). A result of the acquisition of the ECL intensity is illustrated in FIG. 6. Note that an experiment of the electrochemical cyclic voltammetry (CV) is performed by a CHI 630 D electrochemical workstation (Shanghai Shenhua, China). FIG. 6 is a diagram illustrating characterization results of ECL performance of the ECL nanoprobe produced in the example 1.

As illustrated in FIG. 6, an ECL peak does not appear in bare GCE. However, a strong anodic ECL signal is acquired at +1.1 V in COOH—Ru@SiO₂/GCE, indicating that COOH—Ru@SiO₂ can react with TPrA to generate an ECL signal. Compared with COOH—Ru@SiO₂/GCE, the potential of an ECL start peak of Ab₂-Ru@SiO₂/GCE shifts slightly to positive, the shape of the ECL peak hardly changes, and COOH—Ru@SiO₂ still reacts with TPrA and can generate a strong ECL signal. This result indicates that Ab₂-Ru@SiO₂ has excellent ECL performance and can be used as an ECL nanoprobe.

Note that, in all of the examples, ECL was measured in electrolytic cells of an MPI-A multifunctional electrochemiluminescence analysis system (Xi'an Remex, China) and a standard three-electrode system. A platinum wire electrode, an Ag/AgCl reference electrode, and a glass carbon working electrode (GCE) having a diameter of 5 mM are disposed in the standard three-electrode system. ECL responses are recorded in an ECL detection electrolyte (containing 0.1 M of PH7.4 PBS and 10 mM of TPrA) using the three-electrode system. All ECL measurements are performed at the room temperature and the same applies below.

(3) Characterization of Storage Stability of the ECL Nanoprobe

Storage stability of COOH—Ru@SiO₂ and Ab₂-Ru@SiO₂ prepared in the example 1 was measured as explained below.

COOH—Ru@SiO₂ obtained in the example 1 has good dispersibility in water, has a Tyndall effect, can be dispersed stably in water for five weeks or more, and is not easily agglomerated or precipitated.

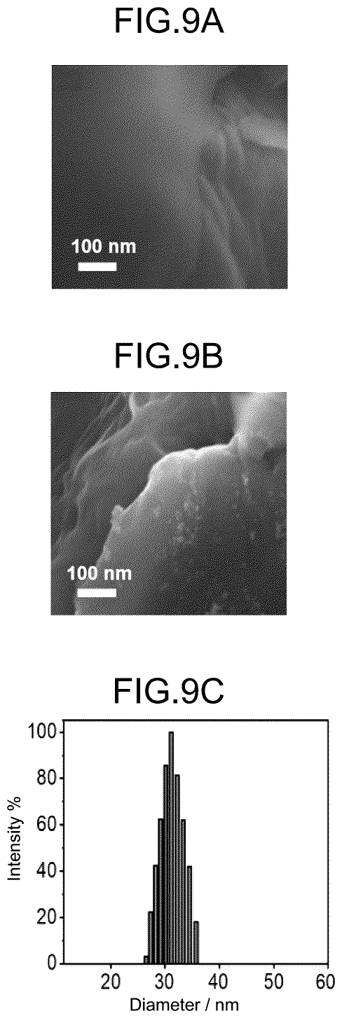

COOH—Ru@SiO₂ was dispersed in water, stored under a 4° C. light blocking condition, and an ECL signal was measured once in every 7 days. Ab₂-Ru@SiO₂ was stored in water containing 1% of BSA under the 4° C. light blocking condition and an ECL signal was measured in every 5 days. Results of the measurement are illustrated in FIGS. 7A to 7B. FIGS. 7A to 7B are diagrams illustrating measurement results of storage stability of the ECL nanoprobe produced in the example 1. FIG. 7A illustrates a measurement results of COOH—Ru@SiO₂. FIG. 7B illustrates a measurement result of Ab₂-Ru@SiO₂.

As illustrated in FIG. 7A, ECL intensity of COOH—Ru@SiO₂ hardly changes within 28 days, indicating that an ECL property is stable. The satisfactory storage stability is assumed to be due to strong electrostatic action between [Ru(byp)₃]²⁺ having positive charges and silica nanoparticles having negative charges. As illustrated in FIG. 7B, ECL intensity of Ab₂-Ru@SiO₂ hardly changes within 20 days, also indicating satisfactory storage stability.

(4) Check of the Number of Ruthenium Molecules and the Number of Secondary Antibodies Bound to Ab₂-Ru@SiO₂

First, from the result of the TEM characterization (the diameter is 30 nm), average volume of single Ru@SiO₂ (V_{Ru@SiO2}):V_{Ru@SiO2}=(4/3) π·r³=(4/3) π·(15×10⁻⁹)³=1.41×10⁻²³ m³ was obtained.

For simplicity, assuming that a pore space is absent in SiO₂ and, since silica density (δ)=2.2 kg/dm³, the weight of single SiO₂ is m_{SiO2}=V_{SiO2}·δ=3.11×10⁻²⁰ kg.

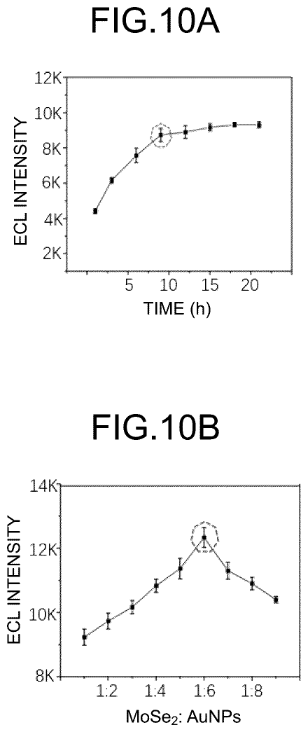

In order to obtain the number of ruthenium molecules bound to single Ru@SiO₂, a quantitative analysis of the Ab₂-Ru@SiO₂ obtained in the example 1 is performed by ultraviolet visible spectra (UV) using a Nanodrop-2000C ultraviolet visible spectrophotometer (Thermo, USA). Results of the quantitative analysis are illustrated in FIGS. 8A to 8C. FIGS. 8A to 8C are diagrams illustrating analysis results of the number of ruthenium molecules and the number of secondary antibodies bound on the ECL nanoprobe produced in the example 1. FIG. 8A illustrates ultraviolet visible spectra of [Ru(byp)₃]2+ standard solutions having different concentrations and an ECL nanoprobe. “a” indicates an absorbance curve of Ru@SiO₂, “b” indicates an absorbance curve of Ab₂-Ru@SiO₂, and other lines indicate absorbance curves of [Ru(byp)₃]2+ having the different concentrations. FIG. 8B illustrates a concentration calibration curve of [Ru(byp)₃]2+ at 457 nm. FIG. 8C illustrates a calibration curve of a BSA standard product at 620 nm.

As illustrated in FIG. 8A, absorbance A at 457 nm of 100 μg/mL of Ru@SiO₂ is 0.26. From the concentration calibration curve of [Ru(byp)₃]²⁺ at 457 nm in FIG. 8B, concentration of [Ru(byp)₃]²⁺ in 100 μg/mL of Ru@SiO₂ was obtained as 12.36 μg/mL. Consequently, the number N_{Ru@SiO2} of 1 mL of 100 μg/mL Ru@SiO₂=m_{total SiO2}/m_{SiO2}=3.22×10¹² and the number N_{[Ru(byp)3]2+} of [Ru(byp)₃]²⁺=NA·n=NA·m_{Ru}/M_{Ru}=9.93×10¹⁵ (M_{Ru}=748.62).

Therefore, the number of [Ru(byp)₃]²⁺ molecules included in the single Ru@SiO₂ is approximately N_{[Ru(byp)3]2+}/N_{Ru@SiO2}=3×10³.

As a result of measuring absorbance at 620 nm of Ab₂ in Ab₂-Ru@SiO₂ obtained in the example 1 using a Bradford method protein quantification kit for a plate reader, absorbance A620 of Ab₂ in Ab₂-Ru@SiO₂ is 0.63.

From the concentration calibration curve of the BSA standard product at 620 nm in FIG. 8C, concentration of Ab₂ in Ab₂-Ru@SiO₂ was obtained as 75.49 μg/mL. Consequently, the number N_{Ab2} of Ab₂ in 1 mL of Ab₂-Ru@SiO₂=NA·n=NA·m_{Ab2}/M_{Ab2}=3×10¹⁴ (M_{Ab2}=150 KD=1.5×10⁵ g/mol).

Ultraviolet quantification of the prepared Ab₂-Ru@SiO₂ after 5-fold dilution thereof was performed. Absorbance A_{Ab2-Ru@SiO2} at 457 nm=0.53. Since absorbance A at 457 nm of 100 μg/mL of Ru@SiO₂=0.26 and the number N_{Ru@SiO2} of Ru@SiO₂=3×10¹²/mL, the number of Ru@SiO₂ in Ab₂-Ru@SiO₂ after 5-fold dilution of 1 mL is N₂=6×10¹²/mL.

It is possible to estimate that the number N₃ of Ab₂ in Ab₂-Ru@SiO₂ after 5-fold dilution of 1 mL=6×10¹³ and the number of antibodies bound to single Ru@SiO₂=N₃/N₂=6×10¹³/6×10¹²=10.

From the above, one Ru@SiO₂ can contain approximately 3×10³ [Ru(byp)₃]²⁺ molecules and the one Ru@SiO₂ can bind approximately 10 antibodies. Therefore, single Ru@SiO₂ can carry up to more than a thousand [Ru(byp)₃]²⁺ molecules to amplify a signal.

### Example 3: ECL Detection Method for Methylated DNA

First, the ECL sensor in the present embodiment is produced and ECL detection is performed as explained below.

(1) Preparation of an Electrocatalyst (MoSe₂) and Metal Nanoparticles (Au NPs)

Preparation of AuNPs

49 mL of deionized water is added to a three-necked flask and heated and boiled. Thereafter, 2 mL of citric acid (2 wt %) is added to the deionized water immediately after 1 mL of HAuCl₄ (1 wt %) is added thereto. The deionized water is stirred for 15 minutes, stopped being heated until a color turns to watermelon red, naturally cooled to the room temperature, and finally stored at 4° C.

Preparation of MoSe₂

0.5 mmol of (NH₄)₂MoO₄ and 1.0 mmol of Se powder are added to 18 mL of an oleic acid/ethanol (1:1 (V/V)) liquid mixture, mixed, and transferred to a high pressure vessel of a polytetrafluoroethylene liner. The autoclave is sealed and (NH₄)₂MoO₄ and the Se powder are placed in an oven, heated at 160 to 200° C. for 72 h, and cooled to the room temperature. Thereafter, a product is centrifuged and cleaned three times by ethanol (10,000 rpm, 10 minutes) and dried in vacuum at 60° C. for 12 h. Further, the product is annealed at 500° C. for 1 to 2 h using an argon gas to remove unreacted selenium powder and surfactant oleic acid on the surface. Finally, 3.5 mg of MoSe₂ was weighed, put in 1 mL of deionized water, ultrasonically exfoliated (35 HZ) for 10 h, and centrifuged again (12,000 rpm, 10 minutes) to remove a trace amount of impurities and MoSe₂ not completely exfoliated, and a supernatant was taken to obtain a MoSe₂ solution (3.5 mg/mL).

(2) Sensor Construction

First, the 3.5 mg/mL of produced MoSe₂/AuNPs solution and 0.50 mg/mL of AuNPs are mixed at a volume ratio of 1:6 and ultrasonically vibrated for 30 minutes to produce a MoSe₂/AuNPs solution. 10 μL of the MoSe₂/AuNPs solution is placed on the surface of a polished glass carbon electrode and dried under vacuum. Subsequently, 10 μL of 30 nM mercapto-modified capture nucleic acid (hereafter also described as SH-DNA) was dissolved in a B&W buffer (containing 0.01 M of Tris-HCl, 1 mM of EDTA, and 2 M of NaCl and having pH of 7.5) and dropped onto the surface of the electrode and incubated at 37° C. and 550 rpm for 50 minutes. After the capture nucleic acid was washed away clean, 10 μL of 1 mM MCH was further dropped onto the surface of the electrode and incubated at 37° C. and 500 rpm for 30 minutes. After MCH was washed away clean, 10 μL of 2% BSA was further dropped onto the surface of the electrode and incubated for 30 minutes and washed away. Thereafter, an ECL sensor was obtained.

(3) Detection of a Target Nucleic Acid

A PCDHGB7 gene methylated in this example is set as a target nucleic acid sequence. Note that all of DNA sequences used in this example were synthesized and purified by Shanghai Sangon Biotech. The sequences in use are specifically illustrated in Table 1.

TABLE 1

| Name | Sequence (5' -> 3') |
|---|---|
| SH-DNA | SH-AGCAGCAGCAAAGGAAATAGTACCTG |
| Target nucleic acid | CCAGCTGCGCGCAGAGGCGCCGGGCCGGC |
| (without methylation | CCGCGGCAGGTACTATTTCCTTTGCTGCT |
| modification) | GC |
| 0x5mC-PCDHGB7 (T₀) | |
| Target nucleic acid | AGCTGC(M)GC(M)GCAGAGGC(M)GCC |
| (with methylation | (M)GGGCC(M)GGCCC(M)GC(M)GGCAG |
| modification) | GTACTATTTCCTTTGCTGCTGCT |
| 7x5mC-PCDHGB7 (T_{5mC}) | |
| 7x5mC-RASSF1A (T_{P}) | GCTTTGC(M)GGTC(M)GCC(M)GTC(M) |
| | GTTGTGGCC(M)GTCC(M)GGGGTGGGGT |
| | GTGAGGAGGGGAC(M)GAAGGAG |

In Table 1, C(M) indicates 5-methylcytosine (5-mC) and SH-DNA has a 3′ end sequence of target nucleic acid PCDHGB7 genes (T) to be detected and 28 complementary bases.

A target nucleic acid is detected as explained below. That is, 10 μL of a target nucleic acid solution (prepared in 10 mM of PBS) is dropped into the produced ECL sensor explained above, incubated for 30 minutes (37° C., 500 rpm), washed, and thereafter 10 μL of 3 μg/mL Ab-5mC is dropped, incubated for 40 minutes, and subsequently, the surface of a sensing electrode is washed and 10 μL of 8 μg/mL Ab₂-Ru@SiO₂ is dropped and incubated for 30 minutes. After being washed, the sensing electrode configured a three-electrode system together with Ag/AgCl and a Pt wire electrode and an ECL response was recorded in a detection solution (containing 0.1 M of PH7.4 PBS and 10 mM of TPrA). Cyclic voltammetry was used to perform scanning from 0 to +1.2 V. Scanning speed is 100 mV/s and voltage of a photomultiplier tube (PMT) is set at 600 V.

### Example 4: Characterization of the ECL Sensor Obtained in the Example 3

(1) Characterization of a Sensor Interface

A: Characterization of MoSe₂/AuNPs

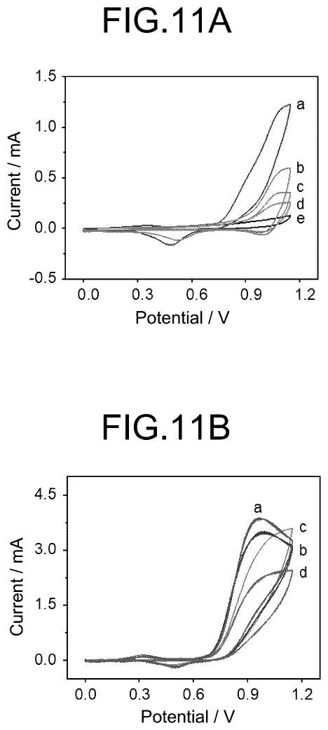

Scanning electron microscope (SEM) images obtained by electronically scanning MoSe₂ and MoSe₂/AuNPs obtained in the example 3 using a SEM (JSM-7800F, JEOL Ltd., Japan) are respectively illustrated in FIGS. 9A and 9B. FIGS. 9A and 9B each illustrate the SEM images of MoSe₂ and MoSe₂/AuNPs. FIG. 9C illustrates a DLS particle size distribution map of AuNPs. FIGS. 9A to 9C illustrate results of nanomaterial characterization at the interface of the ECL sensor produced in the example 3.

As illustrated in FIG. 9A, MoSe₂ clearly exhibits a two-dimensional stacked structure. After MoSe₂ and AuNPs are mixed, as illustrated in FIG. 9B, AuNPs can uniformly cover the surface of an MoSe₂ nanosheet.

A particle size distribution of AuNPs was measured using the dynamic light scattering (DLS) method by using 90 Plus/BI-MAS equipment (Brookhaven, USA). A result of the measurement is illustrated in FIG. 9C. As illustrated in FIG. 9C, a particle size of AuNPs is approximately 30 nm.

### Example 5: Influence of Sensor Interface Construction Conditions on ECL Intensity

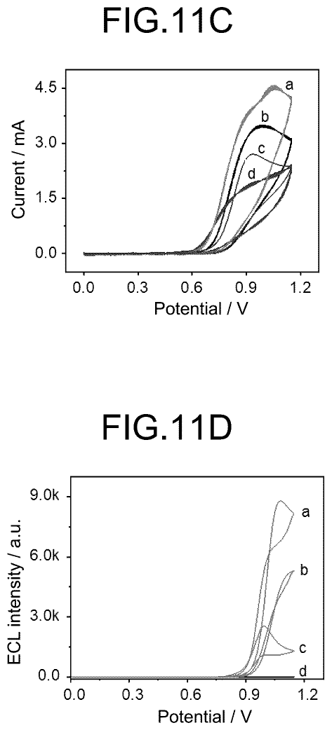

Influence of different construction conditions on ECL intensity was studied using the ECL sensor obtained in the example 3. Results of the study are illustrated in FIGS. 10A to 10B. FIGS. 10A to 10B are diagrams illustrating results of optimizing the construction conditions for a sensor interface. FIG. 10A illustrates the influence of different MoSe₂ exfoliation times on the ECL intensity. FIG. 10B illustrates the influence of different mixing ratios of MoSe₂ and AuNPs on the ECL intensity.

(1) Influence of Different MoSe₂ Exfoliation Times on the ECL Intensity

3.5 mg of MoSe₂ was weighed, put in 1 mL of deionized water and ultrasonically exfoliated (35 HZ) at different times, MoSe₂ solutions obtained at these different ultrasonic exfoliation times were respectively dropped into GCE and dried, thereafter, Ru@SiO₂ was further dropped and dried and thereafter further put in a PBS pH 7.4 detection solution of 10 mM of TPrA, and ECL measurement was performed. A result of the measurement is illustrated in FIG. 10A.

As it is seen from FIG. 10A, as the exfoliation time increased, the ECL intensity increased and reached a maximum stable value at the time of 10 hours. This indicates that a stable two-dimensional sheet layer structure can be obtained after MoSe₂ is exfoliated for 10 hours. Therefore, the MoSe₂ exfoliation time is then set to 10 hours.

(2) Influence of Different Mixing Ratios of MoSe₂ and AuNPs on the ECL Intensity

After 0.5 mg/mL of AuNPs and 1 mL of 3.5 mg/mL MoSe₂ were mixed at different volumes, an electrode was modified and dried, and thereafter Ru@SiO₂ was further dropped, and the ECL detection was performed. A result of the detection is illustrated in FIG. 10B.

As illustrated in FIG. 10B, 6 mL of AuNPs shows the highest ECL response in the electrode modified after being mixed with 1 mL of MoSe₂. Therefore, MoSe₂:AuNPs of 1:6 at a volume ratio is selected to produce a sensor interface.

### Example 6: Preliminary Study of a Mechanism of ECL Interface Enhancement

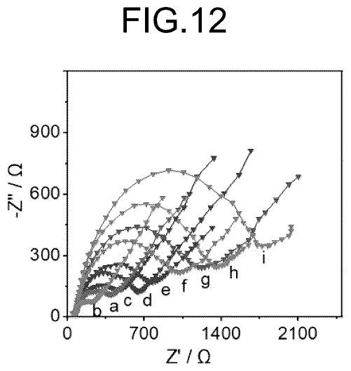

In this example, a mechanism of ECL interface enhancement was studied. Results of the study are illustrated in FIGS. 11A to 11D. FIGS. 11A to 11D are diagrams illustrating results of electrochemical cyclic voltammetry (CV) and ECL measurement in different modified electrodes. FIG. 11A illustrates a measurement result in 0.1 M PBS. In FIG. 11A, a indicates Ru@SiO₂/AuNPs/MoSe₂/GCE, b indicates Ru@SiO₂/AuNPs/GCE, c indicates Ru@SiO₂/MoSe₂/GCE, d indicates Ru@SiO₂/GCE, and e indicates GCE. FIG. 11B illustrates a measurement result in 0.1 M PBS containing 10 mM TPrA. FIGS. 11C and 11D each illustrate CV and ECL intensities in Ru@SiO₂/TPrA systems of different modified electrodes. In FIGS. 11C and 11D, a indicates AuNPs/MoSe₂/GCE, b indicates AuNPs/GCE, c indicates MoSe₂/GCE, and d indicates GCE.

An electrochemical cyclic voltammetry (CV) experiment of Bare GCE, GCE with drops of Ru@SiO₂ (Ru@SiO₂/GCE), MoSe₂-modified GCE with drops of Ru@SiO₂ (Ru@SiO₂/MoSe₂/GCE), AuNPs-modified GCE with drops of Ru@SiO₂ (Ru@SiO₂/AuNPs/GCE), and AuNPs/MoSe₂-modified GCE with drops of Ru@SiO₂ (Ru@SiO₂/AuNPs/MoSe₂/GCE) was performed in 0.1 M of pH 7.4 PBS. A result of the experiment is illustrated in FIG. 11A.

As illustrated in FIG. 11A, in all of different electrodes with drops of Ru@SiO₂, an oxidation peak of Ru(bpy)₃³⁺ appears at potential of 1.1 V and modification with MoSe₂ or AuNPs alone can increase the oxidation peak of Ru(bpy)₃³⁺. However, co-modification of MoSe₂ and AuNPs can synergistically promote the oxidation of Ru(bpy)₃³⁺ and exhibit a higher oxidation current.

Response statuses of the bare GCE, MoSe₂-modified GCE (MoSe₂/GCE), AuNPs-modified GCE (Au NPs/GCE), and AuNPs/MoSe₂-modified GCE (Au NPs/MoSe₂/GCE) explained above to TPrA were further examined. A result of the examination is illustrated in FIG. 11B.

As it is seen from FIG. 11B, the co-modification of MoSe₂ and AuNPs can synergistically promote the oxidation of TPrA. Therefore, in the Ru@SiO₂/TPrA system, AuNPs/MoSe₂/GCE also shows the highest oxidation current (FIG. 11C) and the largest ECL response (FIG. 11D).

As it is seen from the above results, co-modification of the interface with AuNPs and MoSe₂ clearly has an ECL signal enhancing effect on the Ru@SiO₂/TPrA system. In other words, the ECL-based electrode has an interface co-modified by an electrocatalytic solution and metal nanoparticles.

### Example 7: ECL Detection in the Present Embodiment

In this example, ECL detection is studied using the ECL sensor obtained in the example 3.

(1) Electrochemical Characterization

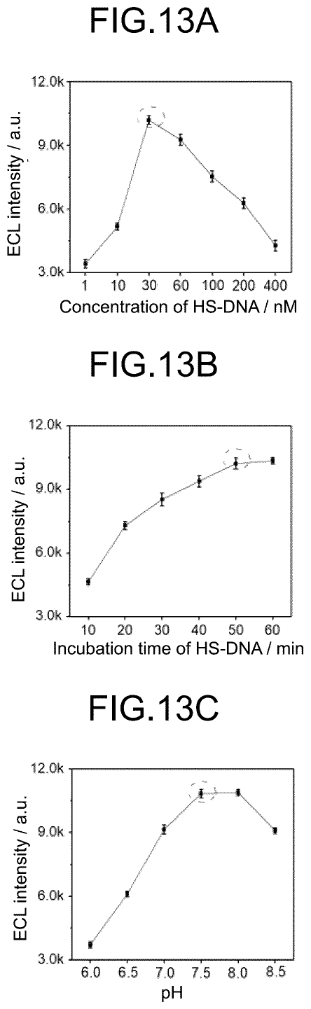

From the bare GCE, electrochemical impedance spectra (EIS) were measured for modification and reaction by steps in the GCE. A result of the measurement is illustrated in FIG. 12. FIG. 12 is a diagram illustrating an EIS characterization result in 0.1 M of KCl containing 5 mM of [Fe(CN)₆]^{3-/4-} of different modified electrodes. In FIG. 12, a indicates the bare GCE, b indicates AuNPs/MoSe₂/GCE, c indicates MoSe₂/GCE, d indicates SH-DNA/AuNPs/MoSe₂/GCE, e indicates MCH/SH-DNA/AuNPs/MoSe₂/GCE, f indicates BSA/MCH/SH-DNA/AuNPs/MoSe₂/GCE, g indicates T_{5mc}/BSA/MCH/SH-DNA/AuNPs/MoSe₂/GCE, h indicates Ab-5mC/T_{5mc}/BSA/MCH/SH-DNA/AuNPs/MoSe₂/GCE, and i indicates Ab₂-Ru@SiO₂/Ab-5mC/T_{5mc}/BSA/MCH/SH-DNA/AuNPs/MoSe₂/GCE. Here, the electrochemical impedance spectra (EIS) were tested by DH 7000 (Jiangsu Donghua, China).

As illustrated in FIG. 12, the impedance gradually increases according to the modification of the GCE by AuNPs/MoSe₂, SH-DNA, MCH, BSA, T5mc, Ab-5mc, and Ab₂-Ru@SiO₂. This indicates that reactions at the steps of the electrode surface has been successful.

(2) Optimization of Detection Conditions

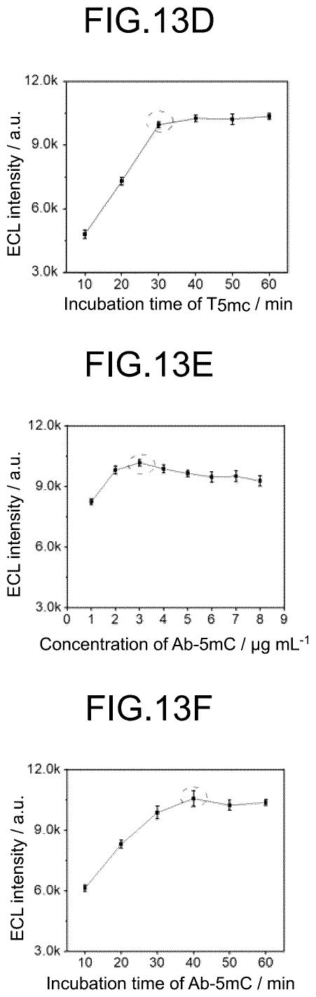

In order to obtain optimal ECL detection performance, ECL response statuses in different detection conditions, for example, pH of an ECL detection solution, concentration and an incubation time of a capture nucleic acid SH-DNA, an incubation time of a target nucleic acid, and concentrations and incubation times of Ab-5mc and Ab₂-Ru@SiO₂ were studied. Results of the study are illustrated in FIGS. 13A to 13H. FIGS. 13A to 13H are diagrams illustrating ECL response statuses of the sensor produced in the example 3 under the different ECL detection conditions. FIG. 13A is a diagram illustrating a relation between different SH-DNA concentrations and ECL intensity. FIG. 13B is a diagram illustrating a relation between incubation times of different SH-DNAs and the ECL intensity. FIG. 13C is a diagram illustrating a relation between detection solutions having different pHs and the ECL intensity. FIG. 13D is a diagram illustrating a relation between different incubation times of T_{5mc} and the ECL intensity. FIG. 13E is a diagram illustrating a relation between different Ab-5mc concentrations and the ECL intensity. FIG. 13F is a diagram illustrating a relation between different incubation times of Ab-5mc and the ECL intensity. FIG. 13G is a diagram illustrating a relation between different Ab₂-Ru@SiO₂ concentrations and the ECL intensity. FIG. 13H is a diagram illustrating a relation between different incubation times of Ab₂-Ru@SiO₂ and the ECL intensity. A principle of selecting the optimal conditions is that conditions corresponding to a maximum value of the ECL response (or a maximum rand period) are set as the optimal conditions.

As illustrated in FIG. 13A, satisfactory ECL intensity can be obtained for all SH-DNA concentrations between 1 nM and 400 nM. Based on the selection principle of the maximum ECL, SH-DNA concentration is preferably 30 nM.

As illustrated in FIG. 13B, satisfactory ECL intensity can be obtained in all cases in which the incubation time of SH-DNA is 10 minutes or longer. The incubation time of SH-DNA is preferably 50 minutes based on the selection principle of the maximum ECL.

As illustrated in FIG. 13C, satisfactory ECL intensity can be obtained in all cases in which pH of the ECL detection solution is between 6.0 and 8.5. Based on the selection principle of the maximum ECL, pH of the ECL detection solution is preferably 7.5.

As illustrated in FIG. 13D, satisfactory ECL intensity can be obtained in all cases in which the incubation time of T_{5mc} is 10 minutes or longer. Based on the selection principle of the maximum ECL, the incubation time of T_{5mC} is preferably 30 minutes.

As illustrated in FIG. 13E, satisfactory ECL intensity is obtained in all cases in which the concentration of Ab-5mc is 1 μg/mL or higher. Based on the selection principle of the maximum ECL, the concentration of Ab-5mc is preferably 3 μg/mL.

As illustrated in FIG. 13F, satisfactory ECL intensity can be obtained in all cases in which the incubation time of Ab-5mc is 10 minutes or longer. Based on the selection principle of the maximum ECL, the incubation time of Ab-5mc is preferably 40 minutes.

As illustrated in FIG. 13G, satisfactory ECL intensity can be obtained in all cases in which the concentration of Ab₂-Ru@SiO₂ is 2 μg/mL or higher. Based on the selection principle of the maximum ECL, the concentration of Ab₂-Ru@SiO₂ is preferably 8 μg/mL.

As illustrated in FIG. 13H, satisfactory ECL intensity can be obtained in all cases in which the incubation time of Ab₂-Ru@SiO₂ is 10 minutes or longer. Based on the selection principle of maximum ECL, the incubation time of Ab₂-Ru@SiO₂ is preferably 30 minutes.

(3) Examination of Detection Performance

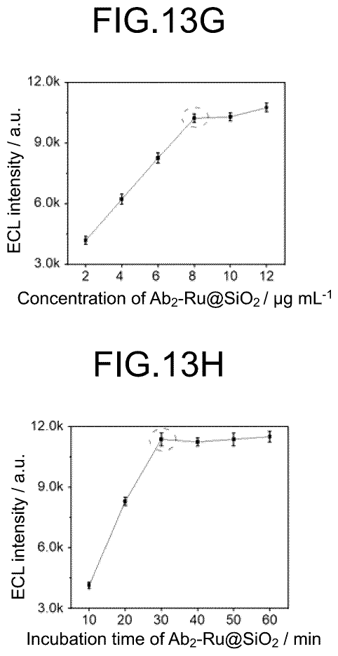

A study for detecting linearity and sensitivity is performed as explained below. That is, methylated DNA (methylated PCDHGB7 gene, T_{5mc}) was detected using the constructed ECL sensor under the preferable conditions explained above. Results of the detection are illustrated in FIGS. 14A to 14B. FIGS. 14A to 14B are diagrams illustrating the results of ECL detection of a target nucleic acid under the optimal conditions. FIG. 14A illustrates ECL response curves for different concentrations T_{5mc}. In FIG. 14A, concentrations of line a to i are respectively 0, 1 fM, 10 fM, 100 fM, 1 pM, 10 pM, 100 pM, 1 nM, and 10 nM. FIG. 14B illustrates a linear relation between ECL intensity and lgC_{T} within a concentration range of 1 fM to 10 nM.

As illustrated in FIG. 14A, it is seen that ECL intensity (I) increases according to an increase in T_{5mc} concentration when ECL detection is performed under conditions of different concentrations T_{5mc}.

FIG. 14B illustrates a logarithmical relation between the ECL intensity (I) and T_{5mc} concentration. It is seen that I and lg CT exhibit a satisfactory linear relation within a range of 1 fM to 10 nM and a correlation coefficient reaches 0.99.

(4) Selectivity Test of Detection

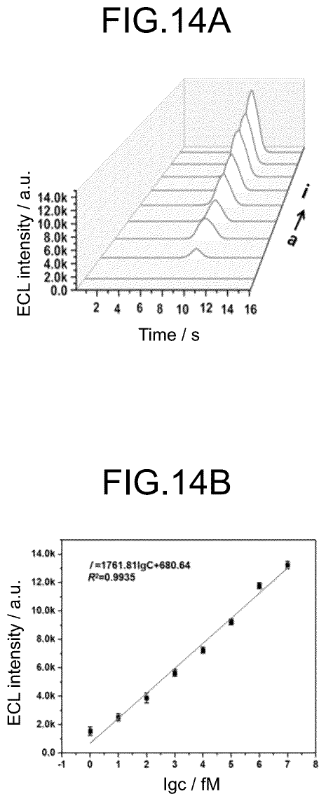

Different sample solutions, a pure background solution (Blank), a solution containing unmethylated PCDHGB7 sequence (T₀), a solution containing methylated RASSF1A sequence (T_{R}) and a solution containing methylated PCDHGB7 sequence (T_{5mC}) were tested by employing the ECL detection method in the present embodiment. Here, all of concentrations of T₀, T_{R} and T_{5mC} are 1 pM. A result of the test is illustrated in FIG. 15. FIG. 15 is a diagram illustrating a result of ECL detection of different sample solutions by the ECL detection method in the present embodiment. In FIG. 15, “Blank” indicates a result of the pure background solution, “T₀” indicates a result of the solution containing unmethylated PCDHGB7 sequence, “T_{R}” indicates a result of the solution containing methylated RASSF1A sequence, and “T_{5mC}” indicates a result of the solution containing methylated PCDHGB7 sequence.

As illustrated in FIG. 15, the sample solution containing T_{5mC} alone has a clear ECL signal and the solutions containing T₀ and T_{R} show a background ECL similar to that of the pure background solution. This indicates that the detection method has satisfactory selectivity for the detection of the methylated PCDHGB7 sequence.

(5) Stability Test of Detection

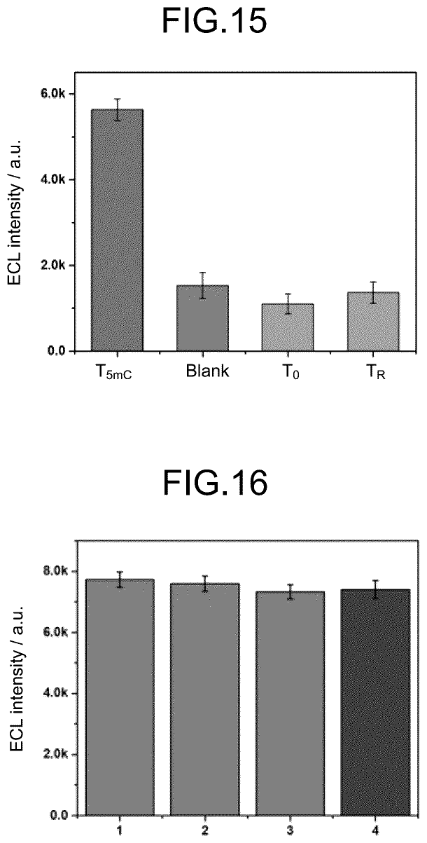

In order to evaluate stability of the detection method in the present embodiment, detection was performed on 10 pM of a target nucleic acid using a plurality of sensors produced in different batches. Here, four sensors (n=4) were produced in each batch. A result of the detection is illustrated in FIG. 16. FIG. 16 is a diagram illustrating a result of repeated detection of 10 pM of the target nucleic acid by the sensors produced in the different batches. In FIG. 16, 1 to 4 respectively indicate the four batches.

As illustrated in FIG. 16, a standard deviation (RSD) of the ECL intensity within 5% indicates a high stability of detection.

The ECL-based electrode, the sensor, and the manufacturing methods for the ECL-based electrode and the sensor, the electrochemiluminescence (ECL) detection method for nucleic acid-specific site modification based on interface enhancement and nanoprobes, and the like have been described above based on the embodiments. However, the invention is not limited thereto. Other aspects obtained by applying various modifications conceivable by those skilled in the art to the embodiments and other aspects constructed by combining a part of the constituent elements in the embodiments are also included in the scope of the invention as long as the aspects do not deviate from the gist of the present invention.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A manufacturing method for an ECL (electrochemiluminescence)-based electrode, comprising a step of mixing an electrocatalytic solution and metal nanoparticles and thereafter dropping a mixed particle suspension onto a surface of an electrode and obtaining a base electrode co-modified by an electrocatalyst-metal nanoparticles.

2. The manufacturing method according to claim 1,
wherein the electrocatalyst is a transition metal compound.

3. The manufacturing method according to claim 1,
wherein the electrocatalyst is molybdenum diselenide (MoSe₂).

4. The manufacturing method according to claim 1,
wherein the metal nanoparticles are at least one of gold (Au) nanoparticles, silver (Ag) nanoparticles, platinum (Pt) nanoparticles, copper (Cu) nanoparticles, cobalt (Co) nanoparticles, iron (Fe) nanoparticles, nickel (Ni) nanoparticles, and multi-alloy nanoparticles thereof.

5. The manufacturing method according to claim 1,
wherein the metal nanoparticles are gold (Au) nanoparticles.

6. The manufacturing method according to claim 1,
wherein the electrode is one of a glass carbon electrode, an ITO (Indium Tin Oxide) electrode, and a screen printing electrode.

7. The manufacturing method according to claim 1,
wherein the electrode is a glass carbon electrode.

8. The manufacturing method according to claim 1,
wherein an exfoliation time of the electrocatalyst is five hours or longer.

9. The manufacturing method according to claim 1,
wherein an exfoliation time of the electrocatalyst is five hours or longer and shorter than 25 hours.

10. The manufacturing method according to claim 1,
wherein a mixing ratio of the electrocatalyst and the metal nanoparticles is 1:1 to 1:9 in a volume ratio of 3.5 mg/mL of MoSe₂ and 0.5 mg/mL of AuNPs.

11. The manufacturing method according to claim 1,
wherein a mixing ratio of the electrocatalyst and the metal nanoparticles is 1:5 to 1:7 in a volume ratio of 3.5 mg/mL of MoSe₂ and 0.5 mg/mL of AuNPs.

12. An ECL-based electrode comprising
an interface co-modified by an electrocatalytic solution and metal nanoparticles.

13. A manufacturing method for an ECL sensor, comprising:
manufacturing an ECL-based electrode, with a manufacturing method for an ECL-based electrode including a step of mixing an electrocatalytic solution and metal nanoparticles and thereafter dropping a mixed particle suspension onto a surface of an electrode and obtaining a base electrode co-modified by an electrocatalyst-metal nanoparticles; and
a step of binding a terminal-modified capture nucleic acid to a surface of the base electrode.

14. The manufacturing method according to claim 13,
wherein the terminal-modified capture nucleic acid is a mercapto-modified capture nucleic acid, an amino-modified capture nucleic acid, or a biotin-modified capture nucleic acid.

15. The manufacturing method according to claim 14,
wherein the terminal-modified capture nucleic acid is a mercapto-modified capture nucleic acid.

16. The manufacturing method according to claim 13,
further comprising a step of blocking, with a blocking agent, the surface of the electrode to which the capture nucleic acid is bound.

17. The manufacturing method according to claim 16,
wherein the blocking agent is at least one of a small molecule blocking agent and a protein blocking agent.

18. The manufacturing method according to claim 17,
wherein the small molecule blocking agent is 6-mercapto-1-hexanol (MCH).

19. The manufacturing method according to claim 17,
wherein the protein blocking agent is bovine serum albumin.

20. An ECL sensor comprising:
an ECL-based electrode having an interface co-modified by an electrocatalytic solution and metal nanoparticles; and
a terminal-modified capture nucleic acid bound to a surface of the ECL-based electrode.

21. An ECL detection method for nucleic acid-specific site modification, comprising:
a first step of dropping a test sample onto a surface of an ECL sensor including an ECL-based electrode having an interface co-modified by an electrocatalytic solution and metal nanoparticles and a terminal-modified capture nucleic acid bound to a surface of the ECL-based electrode and capturing, with the capture nucleic acid, a target nucleic acid on the surface of the ECL sensor;
a second step of dropping an anti-nucleic acid modification antibody and binding the anti-nucleic acid modification antibody to a nucleic acid-specific site on the target nucleic acid;
a third step of adding an ECL nanoprobe and labeling the target nucleic acid with a detection signal, and
a fourth step of immersing the ECL sensor obtained in the third step in a detection solution containing a co-reactive agent to measure an ECL signal.

22. The detection method according to claim 21, wherein
the ECL nanoprobe is metal-doped inorganic oxide nanoparticles modified by a secondary antibody, and
the secondary antibody is affinity-bound to the anti-nucleic acid modification antibody.

23. The detection method according to claim 22,
wherein the inorganic oxide nanoparticles are nanoparticles coated with silicon dioxide, titanium dioxide, zinc oxide, or iron oxide, silicon dioxide nanoparticles, titanium dioxide nanoparticles, zinc oxide nanoparticles, or iron oxide nanoparticles.

24. The detection method according to claim 22,
wherein the inorganic oxide nanoparticles are silicon dioxide nanoparticles.

25. The detection method according to claim 22,
wherein the metal-doped inorganic oxide nanoparticles are silicon dioxide nanoparticles in which the metal-doped inorganic oxide nanoparticles are doped with tris(bipyridine) ruthenium(II) complex ions (Ru(bpy)₃²⁺).

26. The detection method according to claim 21,
wherein the co-reactive agent is tripropylamine.

27. The detection method according to claim 21,
wherein the nucleic acid modification is methylation modification, methylolation modification, or formylation modification of a nucleic acid.

28. The detection method according to claim 21, wherein
pH of the detection solution is 6.0 or higher,
concentration of the capture nucleic acid is 1 nM to 400 nM,
an incubation time of the capture nucleic acid is five minutes or longer,
an incubation time of the target nucleic acid is five minutes or longer,
concentration of the anti-nucleic acid modification antibody is 1 μg/mL or higher,
an incubation time of the anti-nucleic acid modification antibody is five minutes or longer,
concentration of the ECL nanoprobe is 1 μg/mL or higher, and
an incubation time of the ECL nanoprobe is five minutes or longer.

29. The detection method according to claim 21, wherein
pH of the detection solution is 6.5 to 8.5,
concentration of the capture nucleic acid is 20 nM to 100 nM,
an incubation time of the capture nucleic acid is 10 minutes or longer,
an incubation time of the target nucleic acid is 10 minutes or longer,
concentration of the anti-nucleic acid modification antibody is 2 μg/mL or higher,
an incubation time of the anti-nucleic acid modification antibody is 10 minutes or longer,
concentration of the ECL nanoprobe is 2 μg/mL or higher, and
an incubation time of the ECL nanoprobe is 10 minutes or longer.

30. A kit for nucleic acid modification detection used in an ECL detection method for nucleic acid-specific site modification, comprising:
an ECL-based electrode having an interface co-modified by an electrocatalytic solution and metal nanoparticles;
a terminal-modified capture nucleic acid bound to a surface of the ECL-based electrode;
an anti-nucleic acid modification antibody;
an ECL nanoprobe; and
a detection solution.

31. An ECL-based electrode,
co-modified by an electrocatalyst and metal nanoparticles.

32. The ECL-based electrode according to claim 31,
wherein the ECL-based electrode does not contain graphene.


SEQUENCE LISTING (4 sequences)

SEQ ID NO: 1 - DNA, 28 bp, synthetic construct
  source 1..28: mol_type = other DNA
agcagcagcaaaggaaatagtacctgcc

SEQ ID NO: 2 - DNA, 58 bp, synthetic construct
  source 1..58: mol_type = other DNA
agctgcgcgcagaggcgccgggccggcccgcggcaggtactatttcctttgctgctgc

SEQ ID NO: 3 - DNA, 59 bp, synthetic construct
  source 1..59: mol_type = other DNA
  modified_base 7: mod_base = m5c
  modified_base 9: mod_base = m5c
  modified_base 17: mod_base = m5c
  modified_base 20: mod_base = m5c
  modified_base 25: mod_base = m5c
  modified_base 30: mod_base = m5c
  modified_base 32: mod_base = m5c
agctgcgcgcagaggcgccgggccggcccgcggcaggtactatttcctttgctgctgct

SEQ ID NO: 4 - DNA, 60 bp, synthetic construct
  source 1..60: mol_type = other DNA
  modified_base 8: mod_base = m5c
  modified_base 12: mod_base = m5c
  modified_base 15: mod_base = m5c
  modified_base 18: mod_base = m5c
  modified_base 27: mod_base = m5c
  modified_base 31: mod_base = m5c
  modified_base 54: mod_base = m5c
gctttgcggtcgccgtcgttgtggccgtccggggtggggtgtgaggaggggacgaaggag